(19) United States
(12) Patent Application Publication
(10) Pub. No.: US 2010/0313630 A1
(43) Pub. Date: Dec. 16, 2010

(54) MACHINE TOOL AND/OR PRODUCTION MACHINE

(75) Inventor: Andreas Grözinger, Renningen (DE)

Correspondence Address: HENRY M FEIEREISEN, LLC;HENRY M FEIEREISEN, 708 THIRD AVENUE, SUITE 1501, NEW YORK, NY, 10017, US

(73) Assignee: Siemens Aktiengesellschaft, Munchen (DE)

(21) Appl. No.: 12/446,106

(22) PCT Filed: Oct. 16, 2007
(86) PCT No.: PCT/EP2007/061030

§ 371 Date: Apr. 17, 2009

(30) Foreign Application Priority Data
Oct. 19, 2006 (DE) 10 2006 049 328.1

(51) Int. Cl. G01N 33/00

(52) U.S. Cl. 73/23.34

(57) ABSTRACT

The invention relates to a machine tool and/or a production machine, wherein the machine (19) has a sensor (8, 14, 15, 21) for monitoring a machine element of the machine, wherein the sensor (8, 14, 15, 21) detects a chemical substance. The invention creates a machine tool and/or a production machine, with which a machine element of the machine (19) is safely and reliably monitored.

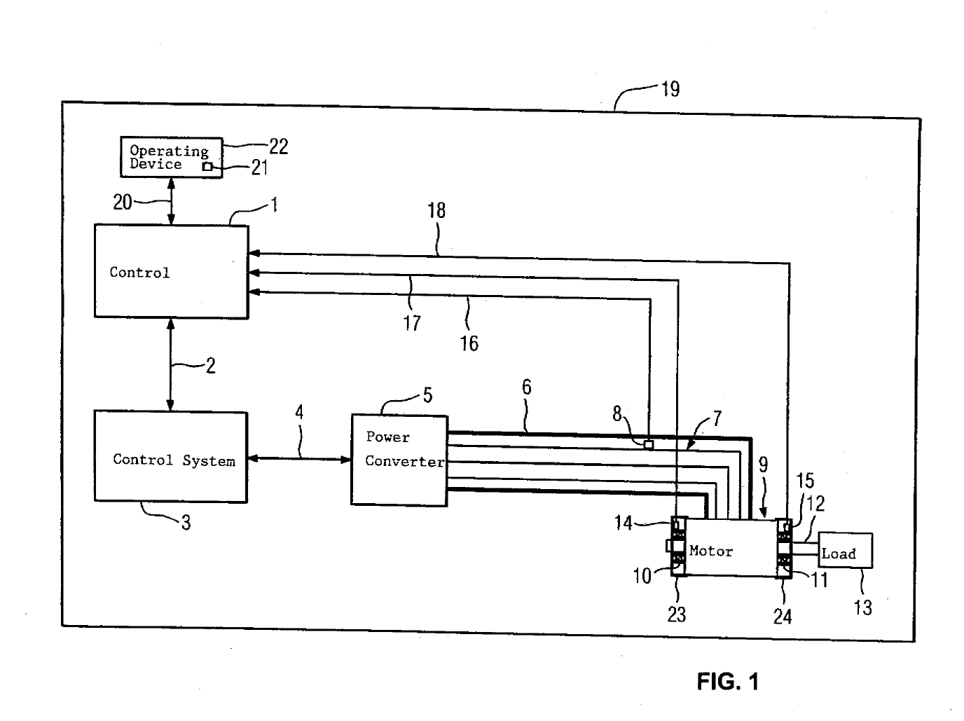

The invention relates to a machine tool and/or a production machine.

In machine tools and/or production machines, faults occur on machine elements, such as for example electric lines, for example in the form of fires caused by scorching of electric lines of the machine. Furthermore, faults occur, for example, in the form of changes in the consistency of lubricants (for example lubricating greases) of bearings, for example of motor shafts of the machine. Furthermore, however, faults may also occur in the form of deposits of chemical substances on machine elements, where they lead to faults.

Until now, such faults in the aforementioned machines could only be detected after considerable damage had already occurred, for example in the form of a fire or bearing damage, for example on a motor shaft of the machine or some other shaft.

It is the object of the invention to provide a machine tool and/or a production machine in which a machine element of the machine is dependably and reliably monitored.

This object is achieved by a machine tool and/or a production machine, wherein the machine has a sensor for monitoring a machine element of the machine, wherein the sensor detects a chemical substance.

Advantageous forms of the invention are provided by the dependent claims.

It proves to be advantageous if the sensor is arranged inside the machine housing, since a machine fault often occurs inside the machine housing.

Furthermore, it proves to be advantageous if the sensor is localized in a cable duct of the machine, since, for example, a fire caused by scorching of an electric line of the machine causes a particularly high concentration of the chemical substance in a cable duct.

Furthermore, it proves to be advantageous if the sensor is formed as an odor sensor. If the sensor is formed as an odor sensor, very sensitive detection of the chemical compound is made possible, with the result that even small concentrations of the chemical substances can be detected.

Furthermore, it proves to be advantageous if the machine element takes the form of an electrical component, in particular the form of an electric line of the machine, wherein the chemical substance develops when the machine element has a fault. This measure allows a faulty machine element to be dependably detected.

It is often the case here that a machine element has a fault in the form of overheating of the machine element, in particular in the form of a fire caused by scorching of the machine element.

Furthermore, it proves to be advantageous if the machine element takes the form of a lubricant of a bearing of the machine, wherein a change in the consistency of the lubricant can be detected by the sensor, by means of detection of the chemical substance. A change in the consistency of a lubricant of a bearing, for example a motor shaft, is generally frequently accompanied by the development of a chemical substance, the concentration of which represents a measure of the degree of the change in the consistency of the lubricant.

Furthermore, it proves to be advantageous if the sensor is arranged in the region of the bearing, in particular on the bearing plate, since the substance can be detected easily there.

Furthermore, it proves to be advantageous if the machine element takes the form of an operating device of the machine, wherein the sensor is localized on and/or in the operating device and depositing of the chemical substance on and/or in the operating device can be detected by means of the sensor. This makes it possible to avoid malfunctions being caused by deposits of substances, for example on and/or in the buttons of the operating device.

In particular if the substance is in the form of graphite, graphite deposits, which occur for example during graphite machining, may lead to faults in the operating device.

An exemplary embodiment of the invention is represented in the drawing and is explained in more detail below.

A schematically represented machine 19, which is formed as a machine tool and/or a production machine, has a control 1, which is connected to a control system 3 via a connection 2 for the exchange of data. The control 1 may in this case be in the form of a numerical control, which calculates setpoint values for a control system 3. Via a connection 4, which serves for the exchange of data, the control system 3 activates a power converter 5, which supplies the electric motor 9 with power by means of a three-phase electric line 7. The line 7 is laid in a cable duct 6. The motor 9 has a motor shaft 12, which rotatably drives a load 13. The motor 12 has two bearings 10 and 11 for the mounting of the motor shaft 12.

The machine 19 has a sensor 8 for the detection of a chemical substance (for example a specific chemical compound), which preferably develops, in particular is formed, when a machine element fault occurs. The fact that the sensor 8 is localized inside the machine housing, and in particular in the cable duct 6, means that a chemical substance developing, for example, as a result of a fire caused by scorching, owing to a scorched plastic insulation of the line 7, can be detected particularly easily by the sensor 8, since it occurs in a high concentration in the cable duct 6. The sensor 8 reports the occurrence of the chemical substance to the control 1, which is indicated by an arrow 16. If the presence of the chemical substance is detected by the sensor 8 and/or if the concentration of the chemical substance exceeds a setpoint value stored in the control 1, an alarm is triggered and, if appropriate, the machine is stopped. Suitable chemical substances for detection are, in particular, chemical compounds which develop when plastics are heated or burned.

Furthermore, within the scope of the exemplary embodiment, the machine 19 additionally has two sensors 14 and 15, wherein a change in the consistency of a lubricant, of the respectively assigned bearings 10 and 11 of the motor 9, is detected by the sensors 14 and 15, by means of the detection of a chemical substance. The lubricant may, for example, be in the form of a lubricating grease. Owing to inadmissibly high loading and/or as a result of aging, a change in the consistency of the lubricant of the bearings 10 and 11 may occur, accompanied by the occurrence of chemical substances, which are detected by the sensors 14 and 15. The occurrence of such a chemical substance is detected by the sensors 14 and 15 and reported to the control 1, which is represented in the figure by two arrows 17 and 18. When such a chemical substance is detected, an alarm is triggered by the control 1 and, if necessary, the machine is stopped. Alternatively, however, the triggering of an alarm and/or the stopping of the machine may also be made dependent on a maximum admissible concentration of the substance being exceeded.

For this purpose, the sensor 14 and the sensor 15 are in this case preferably arranged in the region of the respectively assigned bearings 10 and 11 of the motor 9, in particular on the respectively associated bearing plates 23 and 24, since the concentration of the chemical substance to be detected is greatest there.

Furthermore, within the scope of the exemplary embodiment, the control 1 is connected via a connection 20, which serves for the exchange of data, to an operating device 22 for operating the machine 19. The operating device 22 has a sensor 21, which may be localized on or in the operating device 22, whereby depositing of a chemical substance, such as for example graphite, on and/or in the operating device 22 can be detected by means of the sensor 21.

In this case, the sensors 8, 14, 15 and 21 need not necessarily be designed in such a way that they can only detect a single chemical substance; rather, they may also be formed in such a way that they can detect a number of different chemical substances simultaneously.

It should be mentioned at this point that, within the scope of the invention, a production machine is understood as including particularly a robot.

1-3. (canceled)

4. A machine tool and/or a production machine, comprising:
an operating device; and
a sensor monitoring the operating device and disposed at a location of the operating device selected from the group consisting of on the operating device, in the operating device, and a combination thereof, said sensor detecting a chemical substance, when the chemical substance is deposited on the location.

5. The machine tool and/or production machine of claim 4, wherein the sensor is constructed as an odor sensor.

6. The machine tool and/or production machine of claim 4, wherein the chemical substance is in the form of graphite.
